(19) United States
(12) Patent Application Publication
(10) Pub. No.: US 2012/0046181 A1
(43) Pub. Date: Feb. 23, 2012

(54) Biomarkers for the Diagnosis of Renal Allograft and Kidney Status

(75) Inventors: Jean Harb, Nantes (FR); Maryvonne Hourmant, Nantes (FR); Sandrine Le Roux, Nantes (FR)

(73) Assignees: CHU NANTES, Nantes (FR); INSERM (Institut National de la Sante et de la Recherche Medicale), Paris (FR); UNIVERSITE DE NANTES, Nantes (FR)

(21) Appl. No.: 13/140,494

(22) PCT Filed: Dec. 17, 2009
(86) PCT No.: PCT/EP2009/067398

§ 371 Date: Nov. 7, 2011

(30) Foreign Application Priority Data
Dec. 19, 2008 (EP) 08305988.1

(51) Int. Cl. C40B 30/04; C40B 40/10; G01N 33/566

(52) U.S. Cl. 506/9; 436/501; 435/7.1; 506/18

(57) ABSTRACT

The present invention relates to the identification and use of protein biomarkers with clinical relevance to kidney status and chronic renal injury or disorder. In particular, the invention provides the identity of marker proteins which are recognized by antibodies present in patients suffering from end-stage renal disorder, stable renal transplant, renal transplant glomerulopathy (TG), and interstitial fibrosis and tubular atrophy (IFTA). Methods and kits are described for using these proteins in the study and diagnosis of chronic renal transplant injury, and in the selection and/or monitoring of treatment regimens.

### RELATED APPLICATIONS

The present application claims priority to European Patent Application No. EP 08 305 988.1 filed on Dec. 19, 2008. The European patent application is incorporated herein by reference in its entirety.

### BACKGROUND OF THE INVENTION

Although end-stage disease patients can be treated through other renal replacement therapies such as hemodialysis and peritoneal dialysis, kidney transplantation is generally accepted as the best treatment because it increases patients survival, improves quality of life and presents cost effectiveness. Renal transplantation is by far the most frequently carried out transplantation globally. The World Health Organization estimates that about 66,000 kidney transplants (from living and deceased donors) are performed annually compared to 21,000 liver transplants and 6,000 heart transplants. Renal transplantation is also the most successful and therapeutically advanced of all organ transplant procedures. Recent reports of patient survival in renal transplantation show a 91.3% 1-year and 69.6% 5-year survival for recipients of cadaveric grafts, and a 97.3% 1-year and 80.3% 5-year survival for those receiving living donor transplants (2007 Annual Report of the Scientific Registry of Transplant Recipients and U.S. Organ Procurement and Transplantation Network: Transplant Data 1997-2006). In France, patient survival in renal transplantation shows a 79.1% at 5-years and 62.4% at 10-year survival for recipients of cadaveric grafts, and 89.6% at 5-year and 76.7% at 10-year for those receiving a living donor transplant (2007 Report of “Agence de Biomédecine”: www.agence-biomedicine.fr). With improved immunosuppression and early transplant survival, chronic allograft rejection has become the most prevalent cause of kidney transplant failure. Up to 40% of grafts develop progressive dysfunction after the initial few post-transplant months and ultimately fail within a decade, despite the use of immunosuppressive drugs in doses sufficient to prevent acute rejection.

Several pathophysiological processes contribute to chronic renal transplant injury, including renal transplant glomerulopathy (TG) and interstitial fibrosis and tubular atrophy (IFTA) according to the newly revised Banff classification system (Solez et al., Am. J. Transplant, 2007, 7: 518-526). Renal transplant glomerulopathy is associated with graft dysfunction, hypertension, proteinurea and shortened graft survival. It is a unique pathologic and pathogenic entity distinct from other forms of chronic allograft injury (Suri et al., Am. J. Kidney Dis., 2000, 35: 674-680). Transplant glomerulopathy is characterized by duplication of the glomerular basement membrane, mesangial interposition, and electron luscent widening of the sub-endothelial space with accumulation of flocculent material. The pathogenesis of transplant glomerulopathy is unclear, but, increasingly, investigators hypothesize immune-mediated mechanisms with a strong emphasis on humoral immunity as defined by detection of donor-specific anti-HLA antibodies in patients' serum (Hourmant et al., J. Am. Soc. Nephrol., 2005, 16: 2804-2812) and/or positive C4d staining of the allograft.

The chronic allograft nephropathy (CAN) encompasses all late scarring of the graft due to immunologic and non-immunologic injury. The last Banff classification eliminated the CAN denomination and replaced it with Interstitial Fibrosis and Tubular Atrophy (IFTA). The etiologies of this disease are multiple including chronic hypertension, drug toxicity, viral infection, and chronic obstruction (Solez et al., Am. J. Transplantation, 2007, 7: 518-526).

Biopsies have revealed that renal transplant glomerulopathy and IFTA can develop during the first few months after transplantation (Wavamunno et al., Am. J. Transplantation, 2007, 7: 2757-2768). However, currently available diagnostic methods often fail to detect chronic rejection until late stages of progression, when lesions and damage to the transplant have already occurred. Clinical manifestations such as creatinine and altered resistance indexes on Doppler ultrasonography are not specific. At present, biopsy of the renal allograft is considered as the standard for the diagnosis of chronic rejection. However, percutaneous renal biopsy is a costly, invasive procedure, which carries the risk of procedural complications including allograft thrombosis, sepsis, hematuria, and anuria. Consequently, serial renal biopsies are avoided, which limits the use of this procedure in the follow-up of the disease activity, treatment response and recurrence. This situation explains the need for a non-invasive diagnostic tool that can be used to monitor renal allograft, even before graft dysfunction occurs, and to improve treatment success through early and reliable detection.

Several studies have been undertaken with the goal of identifying biomarkers of chronic renal transplant rejection. Gene expression profiles of chronically rejected renal transplants (Donauer et al., Transplantation, 2003, 76: 539-547; Sarwal, New Engl. J. Medicine, 2003, 349: 125-138), operationally tolerant kidney graft recipients and patients with chronic rejection (WO 2005/070086); kidney biopsies and peripheral blood lymphocytes (Flechner et al., Am. J. Transplantation, 2004, 4: 1475-1489) have been determined using microarrays. A study investigated kidney biopsies performed 6 months post transplant using an Affymetrix GeneChip and identified 10 genes for which expression correlated with the risk of developing chronic rejection defined by biopsy at 12 months post transplant (Scherer et al., Transplantation, 2003, 75: 1323-1330). Recently, efforts are focussing on the characterization of renal rejection by urinary proteomic analysis (Clarke et al., Annals of Surg., 2003, 237: 660-665; WO 2004/030521; Schaub et al., J. Am. Soc. Nephrol., 2004, 15: 219-227; Reichelt et al., Urology, 2006, 67: 472-475; O'Riordan et al., Am. J. Transplant., 2007, 7: 930-340, Thongboonkerd, Proteomics Clin. Appl., 2008, 2: 1413-1421).

### SUMMARY OF THE INVENTION

The present invention relates to improved systems and strategies for the diagnosis of chronic renal transplant rejection. In particular, the invention provides the identity of proteins that reflect clinically relevant processes in dialysis patients with end-stage kidney disorder and in renal transplant patients. More specifically, the invention provides biomarkers that can be used for detecting the presence of antibodies that are selectively indicative of end-stage kidney disorder, renal transplant glomerulopathy, interstitial fibrosis and tubular atrophy (IFTA), and stable renal transplant, in in vitro biological samples (in particular blood samples) obtained from patients. Compared to existing methods of diagnosis, the inventive methods are non-invasive, and the protein profiles disclosed herein constitute a more robust signature of each of the different pathologies, and provide a more reliable basis for the selection and monitoring of appropriate therapeutic regimens.

More specifically, in one aspect, the present invention provides a set of biomarkers indicative of kidney status consisting of RAB20, OAS2, IL1RL1(3), GLRA2, SMAD3, ING4(2), CCL19, CENTD2, ECE1, LGALS3, analogs thereof and antibody-binding fragments thereof; a set of biomarkers indicative of stable renal transplant consisting of ARPC1B, CYP2E1, CDC20, RIT1, PHF21B, RBM34, MS4A6E, C20orf132, LRRFIP1, CKLF(1), analogs thereof and antibody-binding fragments thereof, a set of biomarkers indicative of renal transplant glomerulopathy consisting of PODN, HM13(4), TSC22D3(2), EPN1, COL23A1, GIMAP6, ZNF174, MAP3K11, CRB3(2), PRKCDBP, DCLK1, FEN1, TANK(2), GLRX5, RAB22A, MAEL, FAM71D, TTC35, analogs thereof, and antibody-binding fragments thereof; and a set of biomarkers indicative of IFTA consisting of IL16, MLF1, IFT81(2), DYRK2, NDFIP2, CCNE2, SNX21, TUBB2C, MUC20, ATP1A3, F11R(1), analogs thereof and antibody-binding fragments thereof.

The present invention also provides for the use of at least one of these biomarkers, preferably a plurality of these biomarkers, for the in vitro determination of kidney status or renal transplant status in a subject.

In certain embodiments, the biomarkers are provided immobilized on a solid carrier or support, such as beads or array.

Accordingly, in a related aspect, the present invention provides an array for the in vitro determination of kidney status or renal transplant status in a subject, the array essentially having, attached to its surface, a plurality of biomarkers selected from those described herein.

In another aspect, the present invention provides a method for diagnosing or determining kidney status or renal transplant status in a subject, the method comprising steps of contacting a blood sample obtained from the subject with at least one biomarker for a time and under conditions allowing a biomarker-antibody complex to form between the at least one biomarker and an antibody present in the blood sample; and detecting any biomarker-antibody complex formed. In this method, the at least one biomarker is selected from the group consisting of:

a set of biomarkers indicative of kidney status consisting of RAB20, OAS2, IL1RL1(3), GLRA2, SMAD3, ING4(2), CCL19, CENTD2, ECE1, LGALS3, analogs thereof and antibody-binding fragments thereof,

a set of biomarkers indicative of stable renal transplant consisting of ARPC1B, CYP2E1, CDC20, RIT1, PHF21B, RBM34, MS4A6E, C20orf132, LRRFIP1, CKLF(1), analogs thereof and antibody-binding fragments thereof,

a set of biomarkers indicative of renal transplant glomerulopathy consisting of PODN, HM13(4), TSC22D3(2), EPN1, COL23A1, GIMAP6, ZNF174, MAP3K11, CRB3(2), PRKCDBP, DCLK1, FEN1, TANK(2), GLRX5, RAB22A, MAEL, FAM71D, TTC35, analogs thereof, and antibody-binding fragments thereof, and

a set of biomarkers indicative of IFTA consisting of IL16, MLF1, IFT81(2), DYRK2, NDFIP2, CCNE2, SNX21, TUBB2C, MUC20, ATP1A3, F11R(1), analogs thereof and antibody-binding fragments thereof.

In certain embodiments, the at least one biomarker comprises a plurality of biomarkers, i.e., in other words, the method of the invention comprises steps of: contacting the blood sample with a plurality of biomarkers for a time and under conditions allowing biomarker-antibody complexes to form between the biomarkers and antibodies present in the blood sample; and detecting any biomarker-antibody complex formed.

A blood sample, obtained from the subject and suitable for use in a method of diagnosis of the present invention, may be selected from the group consisting of whole blood, plasma, and serum. Generally, the subject from whom the blood sample is obtained is a dialysis patient or a renal transplant patient.

In certain embodiments, the biomarkers used in the methods of the invention are immobilized on a solid carrier or support, such as beads or array. In certain preferred embodiments, the biomarkers are immobilized on an array.

In the methods provided herein, detection of a biomarker-antibody complex formed between the at least one biomarker and an antibody present in the blood sample may be performed by any suitable method. In certain embodiments, the detection is by immunoassay. For example, the step of detecting a biomarker-antibody complex may comprise contacting the blood sample obtained from the subject with at least one reagent for the detection of the biomarker-antibody complex, such as a labeled anti-human antibody.

In yet another aspect, the present invention provides a kit for the in vitro determination of kidney status or renal transplant status in a subject, the kit comprising at least one biomarker described herein. In certain embodiments, the kit comprises a plurality of biomarkers. Preferably, determination of kidney status or renal transplant status is performed using a blood sample obtained from the subject.

In certain embodiments, the at least one biomarker or the plurality of biomarkers is immobilized on a solid support, such as beads or array. Preferably, the at least one biomarker or the plurality of biomarkers is immobilized on an array.

In certain embodiments, a kit of the present invention further comprises at least one reagent for the detection of a biomarker-antibody complex formed between the at least one biomarker included in the kit and an antibody present in a blood sample obtained from a subject. Such a reagent may be a labeled anti-human antibody.

The kit may further comprise instructions for carrying out any of the methods of diagnosis provided herein.

In certain embodiments, one or both of CCL19 and CDC20 is/are not used in the methods of the present invention and/or is/are not present on an inventive array or an inventive kit.

These and other objects, advantages and features of the present invention will become apparent to those of ordinary skill in the art having read the following detailed description of the preferred embodiments.

### BRIEF DESCRIPTION OF THE DRAWING

FIG. 1 is a table presenting the characteristics of the patients who were tested in the study described in Example 1. Patients 1 to 6 were found to exhibit both TG and IFTA features based on biopsy analysis. Patients 7 to 19 only showed signs of TG. Patients 20 to 29 had IFTA only without C4d deposits in the graft. Patients 30 to 35 were found to have no renal function degradation and had normal biopsies. Dialysis (DIA) patients (who were also tested in the study described in Example 1) are not presented in this table since they had not received kidney graft and therefore did no have any biopsy data.

### DEFINITIONS

Throughout the specification, several terms are employed that are defined in the following paragraphs.

As used herein, the term “subject” refers to a human or another mammal (e.g., primate, dog, cat, goat, horse, pig, mouse, rat, rabbit, and the like) that can undergo kidney transplantation, but may or may not have undergone kidney transplantation. In many embodiments of the present invention, the subject is a human being. In such embodiments, the subject is often referred to as an “individual” or a “patient”. As used herein, the term “renal transplant patient” refers to an individual that has undergone kidney transplantation. The terms “individual” and “patient” do not denote a particular age.

As used herein, the term “diagnosis” refers to a process aimed at determining if an individual is afflicted with a disease or ailment. The terms “diagnosis of renal transplant status” and “determination of renal transplant status” are used herein interchangeably. They refer to a process aimed at one or more of: determining if the renal transplant of a patient is stable, determining if a renal transplant patient has a chronic renal transplant rejection disorder, and determining if the chronic renal transplant rejection disorder is renal transplant glomerulopathy or IFTA. The terms “diagnosis of kidney status” and “determination of kidney status” are used herein interchangeably. They refer to a process aimed at determining if the kidney of a patient is afflicted with end-stage chronic kidney disease.

The term “biological sample” is used herein in its broadest sense. A biological sample is generally obtained from a subject. A sample may be of any biological tissue or fluid with which biomarkers of the present invention may be assayed. Frequently, a sample will be a “clinical sample”, i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample, proteins or nucleic acid molecules extracted from the sample. Processing of a biological sample may involve one or more of: filtration, distillation, extraction, concentration, inactivation of interfering components, addition of reagents, and the like. In preferred embodiments of the invention, the biological sample is (or is derived from) whole blood, serum or plasma obtained from a subject.

The terms “normal” and “healthy” are used herein interchangeably. They refer to an individual or group of individuals who have not undergone kidney transplantation and who have not shown any symptoms of kidney injury, damage or dysfunction. The term “normal” is also used herein to qualify a sample (e.g., a blood sample) obtained from a healthy individual.

In the context of the present invention, the term “control”, when used to characterize a subject, refers to a subject that is healthy, to a patient that has been diagnosed with chronic renal disease (e.g., end-stage chronic kidney disease), or to a renal transplant patient that has been diagnosed with a stable renal transplant, with renal transplant glomerulopathy or with IFTA. The term “control sample” refers to one, or more than one, sample that has been obtained from a healthy subject or from a patient diagnosed with a particular kidney status or renal transplant status as described above.

The terms “biomarker” and “marker” are used herein interchangeably. They refer to a substance that is a distinctive indicator of a biological process, biological event and/or pathologic condition. The term “biomarker of kidney status” refers to a protein selected from the set of proteins provided by the present invention and which is indicative of or specifically recognized by dialysis patients waiting for renal transplantation. The term “biomarker of renal transplant status” refers to a protein selected from the set of protein provided by the present invention and which is indicative of or specifically recognized by renal transplant patients with stable renal transplant, renal transplant glomerulopathy or IFTA.

As used herein, the terms “indicative of kidney status” and “indicative of renal transplant status”, when applied to a process or event, refers to a process or event which is diagnostic of a kidney status or a renal transplant status, such that the process or event is found significantly more often in subjects with a given kidney status or a given renal transplant status than in subjects with a different kidney status or a different renal transplant status (as determined using routine statistical methods). Preferably, a protein biomarker which is indicative of a given kidney status (e.g., end-stage kidney disease) or a given renal transplant status (e.g., stable renal transplant, renal transplant glomerulopathy or IFTA) is recognized by at least 60% of subjects who exhibit the kidney status or the renal transplant status, respectively and is recognized by less than 10% of subjects who do not exhibit the kidney status or the renal transplant status. More preferably, a protein biomarker which is indicative of a given kidney status or of a given renal transplant status is recognized by at least 70%, at least 75%, at least 80%, at least 85%, at least 90%, at least 95% or more patients who exhibit the same kidney status or the same renal transplant status and is recognized by less than 10%, less than 8%, less than 5%, less than 2% or less than 1% subjects who do not exhibit the same kidney status or the same renal transplant status.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is a full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains such as oxidation of sulihydryl groups. Thus, the term “protein” (or its equivalent terms) is intended to include the amino acid sequence of the full-length native protein or a fragment thereof, subject to those modifications that do not significantly change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

The term “protein analog”, as used herein, refers to a polypeptide that possesses a similar or identical function as the protein but need not necessarily comprise an amino acid sequence that is similar or identical to the amino acid sequence of the protein or a structure that is similar or identical to that of the protein. Preferably, in the context of the present invention, a protein analog has an amino acid sequence that is at least about 30%, more preferably at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least bout 99%, identical to the amino acid sequence of the protein.

The term “protein fragment”, as used herein, refers to a polypeptide comprising an amino acid sequence of at least 5 consecutive amino acid residues (preferably at least about: 10, 15, 20, 25, 30, 40, 50, 60, 70, 80, 90, 100, 125, 150, 175, 200, or 250 consecutive amino acid residues) of the amino acid sequence of the protein. The fragment of a protein may or may not possess a functional activity of the protein.

The term “antibody-binding fragment”, when used herein in connection with a protein (in particular a protein biomarker), refers to a fragment of the protein that retains the ability of the protein to bind an antibody to form an antigen-antibody complex. In particular, an antibody-binding fragment of a protein biomarker of the invention retains the ability to bind an antibody specifically found in patients with one type of kidney status or renal transplant status. Suitable antibody-binding fragments of a protein biomarker may be identified by one skilled in the art by simple trials to ascertain their ability to bind kidney status-specific antibodies or renal transplant status-specific antibodies.

The term “homologous” (or “homology”), as used herein is synonymous with the term “identity” and refers to the sequence similarity between two polypeptide molecules or between two nucleic acid molecules. When a position in both compared sequences is occupied by the same base or same amino acid residue, then the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid sequences. Similar residues are conservative substitutions for, or “allowed point mutations” of, corresponding amino acid residues in a reference sequence residue. “Conservative substitutions” of a residue in a reference sequence are substitutions that are physically or functionally similar to the corresponding reference residue, e.g., they have a similar size, shape, electric charge, chemical properties, including the ability to form covalent or hydrogen bonds, or the like. Particularly preferred conservative substitutions are those fulfilling the criteria defined for “accepted point mutation” by Dayhoff et al. (“Atlas of Protein Sequence and Structure”, 1978, Nat. Biomed. Res. Foundation, Washington, D.C., Suppl. 3, 22: 354-352).

The terms “protein array” and “protein chip” are used herein interchangeably. They refer to a substrate surface on which different proteins (or protein fragments) have been immobilized, in an ordered manner, at discrete spots on the substrate. Protein arrays may be used to identify protein/protein interactions (e.g., antigen/antibody interactions), to identify substrates of enzymes, or to identify the targets of biologically active small molecules. The term “microarray” more specifically refers to an array that is miniaturized so as to require microscopic examination for visual evaluation.

The term “protein bead suspension array” refers to a suspension of one or more identifiable distinct particles whereby each particle contains coding features relating to its size and color or fluorescence signature and to which each of the beads of a particular combination is coated with a protein (e.g., a protein biomarker of the invention). Like protein arrays, protein bead suspension arrays may be used to identify protein/protein interactions (e.g., antigen/antibody interactions), to identify substrates of enzymes, or to identify the targets of biologically active small molecules. Examples of bead suspension arrays include thexMAP® bead suspension array (Luminex Corporation).

The terms “labeled”, “labeled with a detectable agent” and “labeled with a detectable moiety” are used herein interchangeably. These terms are used to specify that an entity (e.g., an antibody) can be visualized, for example, following binding to another entity (e.g., a protein biomarker). Preferably, a detectable agent or moiety is selected such that it generates a signal which can be measured and whose intensity is related to the amount of bound entity. In array-based methods, a detectable agent or moiety is also preferably selected such that it generates a localized signal, thereby allowing spatial resolution of the signal from each spot on the array. Methods for labeling proteins and polypeptides are well known in the art. Labeled polypeptides (e.g., antibodies) can be prepared by incorporation of or conjugation to a label, that is directly or indirectly detectable by spectroscopic, photochemical, biochemical, immunochemical, electrical, optical, or chemical means, or any suitable means. Suitable detectable agents include, but are not limited to, various ligands, radionuclides, fluorescent dyes, chemiluminescent agents, microparticles, enzymes, colorimetric labels, magnetic labels, and haptens.

The term “treatment” is used herein to characterize a method that is aimed at (1) delaying or preventing the onset of a disease or condition; or (2) slowing down or stopping the progression, aggravation, or deteriorations of the symptoms of the condition; or (3) bringing about ameliorations or the symptoms of the condition; or (4) curing the condition. A treatment may be administered prior to the onset of the disease, for a prophylactic or preventive action. It may also be administered after initiation of the disease, for a therapeutic action.

### DETAILED DESCRIPTION OF CERTAIN EMBODIMENTS

As mentioned above, the present invention provides biomarkers that can be used for detecting the presence of antibodies that are selectively and specifically indicative of chronic renal disease, stable renal transplant, renal transplant glomerulopathy (TG), and IFTA, in in vitro biological samples obtained from patients. Also provided are methods, arrays and kits for using these biomarkers for determining renal transplant or kidney status in a subject.

### I—Biomarkers

As described in the Examples Section below, the present Applicants have identified protein biomarkers relevant to chronic renal transplant rejection disorders using the ProtoArray Human Protein Microarray commercially available from Invitrogen. This microarray contains more than 8000 human proteins, including proteases/peptidases, secreted proteins, transcription factors, cell death proteins, protein kinases, nuclear proteins, membrane proteins, and metabolism proteins. More specifically, the Applicants have screened samples of serum obtained from dialysis individuals diagnosed with end-stage renal disorder, patients with stable renal transplant, patients diagnosed with renal transplant glomerulopathy, and patients diagnosed with IFTA; and then compared the binding patterns of each group tested with each one of the other three groups. This work led to the identification of highly specific protein biomarkers, i.e., protein biomarkers that are recognized only by one group of the patients tested and that are not recognized by any of the three other groups of patients tested.

The proteins that were selectively recognized by the dialysis patients with end-stage renal disorder (and not by the other groups of patients) are: RAB20 (member RAS oncogene family, which is encoded by a gene whose GenBank Accession Number is NM_{—}017817.1), OAS2 (2′,5′-oligoadenylate synthetase 2 69/71 kDA, which is encoded by a gene whose GenBank Accession Number is BC049215.1); IL1RL1(3) (interleukin 1 receptor-like 1, transcript variant 3, which is encoded by a gene whose GenBank Accession Number is NM_{—}173459.1); GLRA2 (glycine receptor, alpha 2, which is encoded by a gene whose GenBank Accession Number is BC032864.2); SMAD3 (SMAD family member 3, which is encoded by a gene whose GenBank Accession Number is NM_{—}005902); ING4(2) (inhibitor of growth family, member 4, transcript variant 2, which is encoded by a gene whose GenBank Accession Number is NM_{—}198287.1); CCL19 (chemokine (C—C motif) ligand 19, which is encoded by a gene whose GenBank Accession Number is NM_{—}006274.2); CENTD2 (centaurin, delta 2, which is encoded by a gene whose GenBank Accession Number is BC056401.1); ECE1 (endothelin converting enzyme 1, which is encoded by a gene whose GenBank Accession Number is NM_{—}001397.1); and LGALS3 (lectin, galactoside-binding, soluble, 3, which is encoded by a gene whose GenBank Accession Number is BC001120.2).

The proteins that were selectively recognized by the patients with stable renal transplant (and not by the other groups of patients) are: ARPC1B (actin related protein 2/3 complex, subunit 1B, 41 kDa, which is encoded by a gene whose GenBank Accession Number is NM_{—}005720.2); CYP2E1 (cytochrome P450, family 2, subfamily E, polypeptide 1, which is encoded by a gene whose GenBank Accession Number is NM_{—}000773.3); CDC20 (cell division cycle 20 homolog (S. cerevisiae), which is encoded by a gene whose GenBank Accession Number is BC001088.2); RITZ (Ras-like without CAAX 1, which is encoded by a gene whose GenBank Accession Number is NM_{—}006912.4); PHF21B (PHD finger protein 21B, which is encoded by a gene whose GenBank Accession Number is NM_{—}138415.2); RBM34 (RNA binding motif protein 34, which is encoded by a gene whose GenBank Accession Number is NM_{—}015014.1); MS4A6E (membrane-spanning 4-domains, subfamily A, member 6E, which is encoded by a gene whose GenBank Accession Number is NM_{—}139249.2); C20orf132 (chromosome 20 open reading frame 132, which is encoded by a gene whose GenBank Accession Number is BC057767.1); LRRFIP1 (leucine rich repeat (in FLII) interacting protein 1, which is encoded by a gene whose GenBank Accession Number is BC010662.1); and CKLF(1) (chemokine-like factor, transcript variant 1, which is encoded by a gene whose GenBank Accession Number is NM_{—}016951.2).

The proteins that were selectively recognized by the patients with renal transplant glomerulopathy (TG) (and not by the other groups of patients) are: PODN (podocan, which is encoded by a gene whose GenBank Accession Number is BC030608.2); HM13(4) (histocompatibility (minor) 13, transcript variant 4, which is encoded by a gene whose GenBank Accession Number is NM_{—}178582.1); TSC22D3(2) (TSC22 domain family, member 3, transcript variant 2, which is encoded by a gene whose GenBank Accession Number is NM_{—}004089.3); EPN1 (epsin 1, which is encoded by a gene whose GenBank Accession Number is NM_{—}013333.3); COL23A1 (collagen, type XXIII, alpha 1, which is encoded by a gene whose GenBank Accession Number is BC042428.1); GIMAP6 (GTPase, IMAP family member 6, which is encoded by a gene whose GenBank Accession Number is BC060760.1); ZNF174 (zinc finger protein 174, which is encoded by a gene whose GenBank Accession Number is BC000876.1); MAP3K11 (mitogen-activated protein kinase kinase kinase 11, which is encoded by a gene whose GenBank Accession Number is NM_{—}002419.3); CRB3(2) (crumbs homolog 3 (Drosophila), transcript variant 2, which is encoded by a gene whose GenBank Accession Number is NM_{—}139161.3); PRKCDBP (protein kinase C, delta binding protein, which is encoded by a gene whose GenBank Accession Number is BC011585.1); DCLK1 (doublecortin-like kinase 1, which is encoded by a gene whose GenBank Accession Number is NM_{—}004734.2); FEN1 (flap structure-specific endonuclease 1, which is encoded by a gene whose GenBank Accession Number is NM_{—}004111.4); TANK(2) (TRAF family member-associated NFKB activator, transcript variant 2, which is encoded by a gene whose GenBank Accession Number is NM_{—}133484.1); GLRX5 (glutaredoxin 5, which is encoded by a gene whose GenBank Accession Number is NM_{—}016417.2); RAB22A (RAB22A, member RAS oncogene family, which is encoded by a gene whose GenBank Accession Number is NM_{—}020673.2); MAEL (maelstrom homolog (Drosophila), which is encoded by a gene whose GenBank Accession Number is NM_{—}032858.1); FAM71D (family with sequence similarity 71, member D, which is encoded by a gene whose GenBank Accession Number is BC050401.1); and TTC35 (tetratricopeptide repeat domain 35, which is encoded by a gene whose GenBank Accession Number is NM_{—}014673.3).

The proteins that were selectively recognized by the patients with IFTA (and not by the other groups of patients) are: IL16 (interleukin 16 (lymphocyte chemoattractant factor), which is encoded by a gene whose GenBank Accession Number is BC040272.1); MLF1 (myeloid leukemia factor 1, which is encoded by a gene whose GenBank Accession Number is NM_{—}022443.3); IFT81(2) (intraflagellar transport 81 homolog (Chlamydomonas), transcript variant 2, which is encoded by a gene whose GenBank Accession Number is NM_{—}031473.2); DYRK2 (dual-specificity tyrosine-(Y)-phosphorylation regulated kinase 2, which is encoded by a gene whose GenBank Accession Number is BC006375.2); NDFIP2 (Nedd4 family interacting protein 2, which is encoded by a gene whose GenBank Accession Number is BC021988.1); CCNE2 (cyclin E2, which is encoded by a gene whose GenBank Accession Number is BC020729.1); SNX21 (sorting nexin family member 21, which is encoded by a gene whose GenBank Accession Number is BC019823.1); TUBB2C (tubulin, beta 2C, which is encoded by a gene whose GenBank Accession Number is BC029529.1); MUC20 (mucin 20, cell surface associated, which is encoded by a gene whose GenBank Accession Number is BC029267.1); ATP1A3 (ATPase, Na+/K+ transporting, alpha 3 polypeptide, which is encoded by a gene whose GenBank Accession Number is NM_{—}152296.3); and F11R(1) (F11 receptor, transcript variant 1, which is encoded by a gene whose GenBank Accession Number is NM_{—}016946.4).

### Inventive Protein Biomarkers

Accordingly, the present invention provides a set of protein biomarkers that are specifically recognized by dialysis patients diagnosed with end-stage renal disorder, and that can be used for detecting the presence of antibodies that are selectively indicative of end-stage renal disorder in in vitro biological samples (in particular blood samples) obtained from patients. This set of protein biomarkers consists of RAB20, OAS2, IL1RL1(3), GLRA2, SMAD3, ING4(2), CCL19, CENTD2, ECE1, LGALS3, analogs thereof and antibody-binding fragments thereof.

The present invention also provides a set of protein biomarkers that are specifically recognized by renal transplant patients with stable renal implants, and that can be used for detecting the presence of antibodies that are selectively indicative of stable renal transplant in in vitro biological samples (in particular blood samples) obtained from patients. This set of protein biomarkers consists of ARPC1B, CYP2E1, CDC20, RIT1, PHF21B, RBM34, MS4A6E, C20orf132, LRRFIP1, CKLF(1), analogs thereof and antibody-binding fragments thereof.

The present invention also provides a set of protein biomarkers that are specifically recognized by renal transplant patients diagnosed with renal transplant glomerulopathy, and that can be used for detecting the presence of antibodies that are specifically indicative of renal transplant glomerulopathy in in vitro biological samples (in particular blood samples) obtained from patients. This set of protein biomarlers consists of PODN, HM13(4), TSC22D3(2), EPN1, COL23A1, GIMAP6, ZNF174, MAP3K11, CRB3(2), PRKCDBP, DCLK1, FEN1, TANK(2), GLRX5, RAB22A, MAEL, FAM71D, TTC35, analogs thereof, and antibody-binding fragments thereof.

The present invention also provides a set of protein biomarkers that are specifically recognized by renal transplant patients diagnosed with IFTA, and that can be used for detecting the presence of antibodies that are specifically indicative of IFTA in in vitro biological samples (in particular blood samples) obtained from patients. This set of protein biomarkers consists of IL16, MLF1, IFT81(2), DYRK2, NDFIP2, CCNE2, SNX21, TUBB2C, MUC20, ATP1A3, F11R(1), analogs thereof and antibody-binding fragments thereof.

The present invention also provides a set of protein biomarkers that can be used to discriminate between patients with end-stage renal disorder, patients with stable renal transplant, patients with renal transplant glomerulopathy, and patients with IFTA. The set of protein biomarkers consists of RAB20, OAS2, IL1RL1(3), GLRA2, SMAD3, ING4(2), CCL19, CENTD2, ECE1, LGALS3, ARPC1B, CYP2E1, CDC20, RIT1, PHF21B, RBM34, MS4A6E, C20orf132, LRRFIP1, CKLF(1), PODN, HM13(4), TSC22D3(2), EPN1, COL23A1, GIMAP6, ZNF174, MAP3K11, CRB3(2), PRKCDBP, DCLK1, FEN1, TANK(2), GLRX5, RAB22A, MAEL, FAM71D, TTC35, IL16 MLF1, IFT81(2), DYRK2, NDFIP2, CCNE2, SNX21, TUBB2C, MUC20, ATP1A3, F11R(1), analogs thereof and antibody-binding fragments thereof. The pattern of recognition of these proteins by a patient can be used to diagnose a kidney status or a renal transplant status.

Thus, the present invention further provides for the use of at least one of the protein biomarkers described herein to diagnose kidney status or renal transplant status in a subject. The present invention also provides for the use of any combination (i.e., at least two) of the protein biomarkers described herein to diagnose kidney status or renal transplant status in a subject. The present invention also provides for the use of all of the protein biomarkers described herein to diagnose kidney status or renal transplant status in a subject. The present invention further provides for the use of all of the protein biomarkers indicative of stable renal transplant, renal transplant glomerulopathy, and IFTA to diagnose renal transplant status. The present invention further provides for the use of all of the protein biomarkers indicative of end-stage kidney disorder to diagnose kidney status.

Other biomarkers provided by the present invention include nucleic acid molecules comprising polynucleotide sequences coding for the inventive protein biomarkers described herein (or analogs and fragments thereof) and polynucleotides that hybridize with portions of these nucleic acid molecules.

### Preparation of Protein Biomarkers

Protein biomarkers to be used in the methods of diagnosis of the present invention may be prepared by any suitable method, including chemical synthesis and recombinant methods.

For example, the biomarkers of the invention may be prepared using standard chemical methods. Solid-phase peptide synthesis, which was initially described by R. B. Merrifield (J. Am. Chem. Soc. 1963, 85: 2149-2154) is a quick and easy approach to synthesizing peptides and peptidic molecules of known sequences. A compilation of such solid-state techniques may be found, for example, in “Solid Phase Peptide Synthesis” (Methods in Enzymology, G. B. Fields (Ed.), 1997, Academic Press: San Diego, Calif., which is incorporated herein by reference in its entirety). Most of these synthetic procedures involve the sequential addition of one or more amino acid residues or suitable protected amino acid residues to a growing peptide chain. For example, the carboxy group of the first amino acid is attached to a solid support via a labile bond, and reacted with the second amino acid, whose amino group has, beforehand, been chemically protected to avoid self-condensation. After coupling, the amino acid group is deprotected, and the process is repeated with the following amino acid. Once the desired peptide is assembled, it is cleaved off from the solid support, precipitated, and the resulting free peptide may be analyzed and/or purified as desired. Solution methods, as described, for example, in “The Proteins” (Vol. II, 3^{rd} Ed., H. Neurath et al. (Eds.), 1976, Academic Press: New York, N.Y., pp. 105-237), may also be used to synthesize the biomarkers of the invention.

Alternatively, the protein biomarkers provided herein can be produced by recombinant DNA methods. These methods generally involve isolation of the gene encoding the desired protein, transfer of the gene into a suitable vector, and bulk expression in a cell culture system. The DNA coding sequences for the polypeptides of the invention may be readily prepared synthetically using methods known in the art (see, for example, M. P. Edge et al., Nature, 1981, 292: 756-762).

After synthesis, the DNA encoding the desired peptide is inserted into a recombinant expression vector, which may be a plasmid, phage, viral particle, or other nucleic acid molecule-containing vectors or nucleic acid molecule-containing vehicles which, when introduced into an appropriate host cell, contains the necessary genetic elements to direct expression of the coding sequence of interest. Standard techniques well known in the art can be used to insert the nucleic acid molecule into the expression vector. The insertion results in the coding sequence being operatively linked to the necessary regulatory sequences.

Host cells for use in the production of proteins are well known and readily available. Examples of host cells include bacteria cells such as Escherichia coli, Bacillus subtilis, attenuated strains of Salmonella typhimurium, and the like; yeast cells such as Saccharomyces cerevisiae, Schizosaccharomyces pombe, Kluyveromyces strains, Candida, or any yeast strain capable of expressing heterologous proteins; insect cells such as Spodoptera frugiperda; non-human mammalian tissue culture cells such as Chinese Hamster Ovary (CHO) cells, monkey COS cells, and mouse 3T3 cells; and human tissue culture cells such as HeLa cells, HL-60 cells, kidney 293 cells and epidermal S431 cells.

Several expression vectors to produce polypeptides in well known expression systems are commercially available. For example, the plasmids pSE420 (available from Invitrogen, San Diego, Calif.) and pBR322 (available from New England Biolabs, Beverly, Mass.) may be used for the production of the inventive peptides in E. coli. Similarly, the plasmid pYES2 (Invitrogen) may be used for peptide production in S. cerevisiae strains of yeast. The commercially available MacBacR™ kit (Invitrogen) for baculovirus expression system or the BaculoGold™ Transfection Kit available from PharMingen (San Diego, Calif.) may be used for production in insect cells, while the plasmids pcDNA I, pcDNA 3, and pRc/RSV, commercially available from Invitrogen, may be used for the production of the peptides of the invention in mammalian cells such as Chinese Hamster Ovary (CHO) cells.

Other expression vectors and systems can be obtained or produced using methods well known to those skilled in the art. Expression systems containing the requisite control sequences, such as promoters and polyadenylation signals, and preferably enhancers are readily available for a variety of hosts (see, for example, Sambrook et al., Molecular Cloning: A Laboratory Manual, 2^{nd} Ed., 1989, Cold Spring Harbor Press: Cold Spring, N.Y.; and R. Kaufman, Methods in Enzymology, 1990, 185: 537-566).

The expression vector including DNA that encodes the desired protein is used to transform the compatible host cell. The host cell is then cultured and maintained under conditions favoring expression of the desired protein. The protein thus produced is recovered and isolated, either directly from the culture medium or by lysis of the cells. If desired, it can then be characterized by different methods such as Nuclear Magnetic Resonance (NMR) or X-ray crystallography.

As understood by one skilled in the art, a protein biomarker of the present invention may be produced as a fusion protein (i.e., a molecule in which an antibody-binding moiety is linked to a polypeptide entity). Such a polypeptide entity may be selected to confer any of a number of advantageous properties to the resulting fusion protein. For example, the polypeptide entity may be selected to provide increased expression of the recombinant fusion protein. Alternatively or additionally, the polypeptide entity may facilitate purification of the fusion protein for example, by acting as a ligand in affinity purification. A proteolytic cleavage site may be added to the recombinant protein so that the desired sequence can ultimately be separated from the polypeptide entity after purification. The polypeptide entity may also be selected to confer an improved stability to the fusion protein, when stability is a goal. Examples of suitable polypeptide entities include, for example, polyhistidine tags, that allow for the easy purification of the resulting fusion protein on a nickel chelating column. Glutathione-S-transferase (GST), maltose B binding protein, or protein A are other examples of suitable polypeptide entities that can be fused to a protein biomarker of the invention using commercial fusion expression vectors.

Alternatively, protein biomarkers of the invention may be prepared using commercially available proteins.

In certain embodiments, a protein biomarker of the invention is provided immobilized onto a solid carrier or support (e.g., a bead or an array). Methods for immobilizing polypeptide molecules onto a solid surface are known in the art. In particular, the invention provides an array for the diagnosis of kidney status or renal transplant status, consisting essentially of biomarkers described herein, immobilized to its surface. In certain embodiments, the array has, immobilized to its surface, at least one (i.e., one or more than one) of the inventive protein biomarkers that are specific for dialysis patients with end-stage renal disorder, at least one of the inventive protein biomarkers that are specific for patients with stable renal transplant, at least one of the inventive protein biomarkers that are specific for patients with renal transplant glomerulopathy, and at least one of the inventive protein biomarkers that are specific for patients with IFTA. In a particular embodiment, the array has, immobilized to its surface, all of the inventive protein biomarkers that are specific for dialysis patients with end-stage renal disorder, all of the inventive protein biomarkers that are specific for patients with stable renal transplant, all of the inventive protein biomarkers that are specific for patients with renal transplant glomerulopathy, and all of the inventive protein biomarkers that are specific for patients with IFTA. An array of the invention may further comprise various proteins and other controls to allow verifications of reagents background, and detection conditions during probing, assessment of the performance of an assay and/or normalization. Examples of such controls include anti-biotin antibodies which are recognized by the detection IgG antibody, and the Influenza A antigen, which is recognized by approximately 95% of the total population.

The terms “array for diagnosing kidney status or renal transplant status” and “array for determining kidney status or renal transplant status” are used herein interchangeably. They refer to an array for the detection, in an in vitro biological sample, of antibodies that are indicative of one and only one kidney status or renal transplant status: end-stage renal disorder, stable renal transplant, renal transplant glomerulopathy or IFTA.

A protein biomarker may be immobilized by being either covalently or passively bound to the surface of a solid carrier or support. Examples of suitable carrier or support materials include, but are not limited to, agarose, cellulose, nitrocellulose, dextran, Sephadex, Sepharose, carboxymethyl cellulose, polyacrylamides, polystyrene, polyvinyl chloride, polypropylene, gabbros, filter paper, magnetite, ion-exchange resin, glass, polyamine-methyl-vinyl-ether-maleic acid copolymer, amino acid copolymer, ethylene-maleic acid copolymer, nylon, silk, and the like. Immobilization of a protein biomarker on the surface of a solid carrier or support may involve crosslinking, covalent binding or physical adsorption, using methods well known in the art. The solid carrier or support may be in the form of a bead, a particle, a microplate well, an array, a cuvette, a tube, a membrane, or any other shape suitable for conducting a diagnostic method according to the invention (e.g., using an immunoassay). In certain embodiments, immobilization of a protein biomarker to a solid carrier or support includes gel electrophoresis followed by transfer to a membrane (typically nitrocellulose or PVDF) in a process called western blotting (or immunoblot well known in the art).

### II—Diagnosis Methods

The inventive protein biomarkers may be used to detect, in a biological sample obtained from a subject, the presence of antibodies that are indicative of kidney status or renal transplant status.

Accordingly, the present invention provides methods for diagnosing or determining kidney status or renal transplant status in a subject. Such methods comprise contacting a biological sample obtained from the subject (in particular a blood sample) with at least one protein biomarker described herein for a time and under conditions allowing a biomarker-antibody complex to form between the at least one biomarker and an antibody present in the biological sample; and detecting any biomarker-antibody complex formed. Kidney status or renal transplant status may be determined based on the nature of the at least one biomarker used and the detection (or not) of a biomarker-antibody complex.

In certain embodiments, the method comprises contacting a biological sample obtained from the subject (in particular a blood sample) with a plurality of protein biomarkers described herein for a time and under conditions allowing biomarker-antibody complexes to form between the biomarkers and antibodies present in the biological sample; and detecting any biomarker-antibody complex formed. Kidney status or renal transplant status may be determined based on the binding pattern obtained (i.e., the nature of the biomarkers used and the detection (or not) of biomarker-antibody complexes).

The plurality of biomarkers may be any combination of the biomarkers described herein. For example, the plurality of biomarkers may consist of only biomarkers that are indicative of one of the 4 pathologies (e.g., only biomarkers indicative of IFTA or only biomarkers indicative of renal transplant glomerulopathy). Alternatively, the plurality of biomarkers may consist of a combination of biomarkers of the 4 different pathologies. In one embodiment, the plurality of biomarkers consists of all the biomarkers described herein. Alternatively, the plurality of biomarkers may consist of only biomarkers that are indicative of renal transplant status (i.e., biomarkers that are indicative of stable renal transplant, biomarkers that are indicative of renal transplant glomerulopathy and biomarkers that are indicative of IFTA).

### Biological Samples

The methods of diagnosis of the present invention may be applied to the study of any type of biological samples allowing one or more inventive biomarkers to be assayed. Examples of suitable biological samples include, but are not limited to, blood samples, and urine. Preferred biological samples are blood samples, i.e., whole blood, serum or plasma. Biological samples used in the practice of the invention may be fresh or frozen samples collected from a subject, or archival samples with known diagnosis, treatment and/or outcome history. Biological samples may be collected by any non-invasive means, such as, for example, by drawing blood from a subject.

In preferred embodiments, the inventive methods are performed on the biological sample itself without, or with limited, processing of the sample.

However, alternatively, the inventive methods may be performed on a protein extract prepared from the biological sample. In this case, the protein extract preferably contains the total protein content. Methods of protein extraction are well known in the art (see, for example, “Protein Methods”, D. M. Bollag et al., 2^{nd} Ed., 1996, Wiley-Liss; “Protein Purification Methods: A Practical Approach”, E. L. Harris and S. Angal (Eds.), 1989; “Protein Purification Techniques: A Practical Approach”, S. Roe, 2^{nd} Ed., 2001, Oxford University Press; “Principles and Reactions of Protein Extraction, Purification, and Characterization”, H. Ahmed, 2005, CRC Press: Boca Raton, Fla.). Various kits can be used to extract proteins from bodily fluids and tissues. Such kits are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kit(s) most appropriate for a particular situation.

### Detection of Biomarker-Antibody Complexes

The diagnostic methods of the present invention generally involve detection of at least one complex formed between a protein biomarker and an antibody present in a biological sample. In the practice of the invention, detection of such a biomarker-antibody complex may be performed by any suitable method (see, for example, E. Harlow and A. Lane, “Antibodies: A Laboratories Manual”, 1988, Cold Spring Harbor Laboratory: Cold Spring Harbor, N.Y.).

For example, detection of a biomarker-antibody complex may be performed using an immunoassay. A wide range of immunoassay techniques is available, including radioimmunoassays, enzyme immunoassays (EIA), enzyme-linked immunosorbent assays (ELISA), and immunofluorescence immunoprecipitation. Immunoassays are well known in the art. Methods for carrying out such assays as well as practical applications and procedures are summarized in textbooks. Examples of such textbooks include P. Tijssen, In: Practice and theory of enzyme immunoassays, eds. R. H. Burdon and v. P H Knippenberg, Elsevier, Amsterdam (1990), pp. 221-278 and various volumes of Methods in Enzymology, Eds. S. P. Colowick et al., Academic Press, dealing with immunological detection methods, especially volumes 70, 73, 74, 84, 92 and 121. Immunoassays may be competitive or non-competitive.

For example, any of a number of variations of the sandwich assay technique may be used to perform an immunoassay. Briefly, in a typical sandwich assay applied to the detection of antibodies indicative of a kidney status or a renal transplant status according to the present invention, an unlabeled protein biomarker is immobilized on a solid substrate and the sample to be tested is brought into contact with the bound biomarker for a time and under conditions allowing formation of a biomarker-antibody complex. Following incubation, a secondary antibody that is labeled with a detectable moiety and that specifically recognizes antibodies from the species tested (e.g., an anti-human IgG for human subjects) is added and incubated under conditions allowing the formation of a ternary complex between any biomarker-bound antibody and the labeled secondary antibody. Any unbound material is washed away, and the presence of any antibody indicative of a kidney status or a renal transplant status is determined by observation of the signal directly or indirectly produced by the detectable moiety. Variations in this assay include an assay in which both the biological sample and the labeled secondary antibody are added simultaneously to the immobilized biomarker (or biomarkers).

The secondary antibody may be labeled with any suitable detectable moiety, i.e., any entity which, by its chemical nature, provides an analytical identifiable signal allowing detection of the ternary complex, and consequently detection of the biomarker-antibody complex.

Detection may be either qualitative or quantitative. Methods for labeling biological molecules such as antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

The most commonly used detectable moieties in immunoassays are enzymes and fluorophores. In the case of an enzyme immunoassay (EIA), an enzyme such as horseradish peroxidase, glucose oxidase, beta-galactosidase, alkaline phosphatase, and the like, is conjugated to the second antibody, generally by means of glutaraldehyde or periodate. The substrate to be used with the specific enzymes is generally chosen for the production of a detectable color change, upon hydrolysis of the corresponding enzyme. In the case of immunofluorescence, the second antibody is chemically coupled to a fluorescent moiety without alteration of its binding capacity. After binding of the fluorescently labeled antibody to the biomarker-antibody complex and removal of any unbound material, the fluorescent signal generated by the fluorescent moiety is detected, and optionally quantified. Alternatively, the second antibody may be labeled with a radioisotope, a chemiluminescent moiety, or a bioluminescent moiety.

### Determination of Kidney Status or Renal Transplant Status

In the methods of the present invention, detection of a biomarker-antibody complex is indicative of the presence of specific antibodies in the biological sample tested and is therefore indicative of the kidney status or renal transplant status of the subject from whom the biological sample has been obtained. Thus, methods of the present invention may be used for determining (or diagnosing) kidney status (i.e., end-stage kidney disorder) or renal transplant status (i.e., stable renal transplant, renal transplant glomerulopathy, or IFTA). In particular, methods of the invention may be used for testing subjects after renal transplant as a way of monitoring renal transplant status.

It will be appreciated by one skilled in the art that diagnosis (or determination) of kidney status or renal transplant status may be made solely on the results provided by a method described herein. Alternatively, a physician may also consider other clinical or pathological parameters used in existing methods to diagnose renal transplant status. Thus, results obtained using methods of the present invention may be compared to and/or combined with results from other tests, assays or procedures performed for the diagnosis of kidney status or renal transplant status. Such comparison and/or combination may help provide a more refine diagnosis.

Alternatively or additionally, results from diagnosis methods of the present invention may be used in combination with results from one or more assays that employ other biomarkers of renal transplant status. Thus, in certain embodiments, diagnostics of renal transplant status may be based on results from a method of the invention and results from one or more additional assays using different biomarkers.

### Selection of Appropriate Treatment

The biomarkers and methods of diagnostic provided by the present invention have the potential to radically change the way in which transplant patients are managed. The methods are non-invasive, and have no associated morbidity or mortality. They require only small volumes of samples, and are highly cost-effective when compared to other clinical and biochemical modalities such as biopsies. They are rapid, and can easily be repeated over time allowing a frequent surveillance and monitoring of renal transplant status and function. In addition, the assays have the potential for development of automated analysis for the clinical laboratory.

Thus, using methods described herein, skilled physicians may select and prescribe treatments adapted to each individual patient based on the diagnosis (or determination) of the renal transplant status. In particular, the present invention provides physicians with a means to diagnose early renal transplant glomerulopathy, which will allow for early treatment, when intervention is likely to have its greatest effect. Frequent non-invasive renal transplant monitoring according to the invention could allow for immunosuppression doses to be increased or decreased according to specific individual needs. For example, immunosuppression could be lowered, even gradually weaned off, in patients with stable renal transplant. Decision to reduce or eliminate immunosuppressive drugs could be made with a strategy to safely monitor the results before clinically apparent changes in kidney function occur. Alternatively, for patients in whom early rejection is detected, immunosuppression could be increased until a satisfactory response is achieved. Adequate response to treatment and resolution of rejection could be rapidly verified, preventing over-immunosuppression and its associated consequences.

The methods of the present invention may also find applications in the development of new therapeutics, such as new immunosuppressive drugs, for the management or treatment of renal transplant patients.

### III—Kits

In another aspect, the present invention provides kits comprising materials useful for carrying out the diagnostic methods of the invention. The diagnostic procedures described herein may be performed by clinical laboratories, experimental laboratories, or practitioners. The invention provides kits which can be used in these different settings.

In certain embodiments, an inventive kit comprises at least one biomarker described herein, preferably in an amount that is suitable for detecting antibodies in a biological sample, and optionally, instructions for using the kit according to a method of the invention. The biomarkers may or may not be immobilized on a substrate surface (e.g., beads, array and the like).

In preferred embodiments, an inventive kit comprises a plurality of biomarkers described herein, preferably each one in an amount that is suitable for detecting antibodies in a biological sample. The plurality of biomarkers may be any combination of the biomarkers described herein. For example, kits that are specifically designed for the diagnostic of one of the 4 pathologies will preferably comprise only biomarkers that are indicative of the pathology of interest (e.g., only biomarkers indicative of IFTA, or only biomarkers indicative of renal transplant glomerulopathy). Alternatively, kits that are designed for the determination of kidney status or renal transplant status will preferably comprise a combination of biomarkers of the 4 different pathologies. In one embodiment, the plurality of biomarkers included in the kit will consist of all the biomarkers described herein. Alternatively, kits that are designed for the determination of renal transplant status only will preferably comprise a combination of biomarkers that are indicative of renal transplant status (i.e., biomarkers that are indicative of stable renal transplant, biomarkers that are indicative of renal transplant glomerulopathy and biomarkers that are indicative of IFTA).

In addition, an inventive kit may further comprise at least one reagent for the detection of a biomarker-antibody complex formed between a biomarker included in the kit and an antibody present in a biological sample obtained from a patient. Such a reagent may be, for example, a labeled antibody that specifically recognizes antibodies from the species tested (e.g., an anti-human IgG), as described above. If the biomarkers are provided attached to the surface of an array, a kit of the invention may comprise only one reagent for the detection of biomarker-antibody complexes (e.g., a fluorescently-labeled anti-human antibody).

Depending on the procedure, the kit may further comprise one or more of: extraction buffers and/or reagents, western blotting buffers and/or reagents, immunodetection buffers and/or reagents, labeling buffers and/or reagents, and detection means. Protocols for using these buffers and reagents for performing different steps of the procedure may be included in the kit.

The different reagents included in an inventive kit may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, a kit further comprises instructions for using its components for the diagnosis of kidney status or renal transplant status in a subject according to a method of the invention. Instructions for using the kit according to methods of the invention may comprise instructions for processing the biological sample obtained for the subject and/or for performing the test, and/or instructions for interpreting the results. A kit may also contain a notice in the form prescribed by a governmental agency regulating the manufacture, use or sale of pharmaceuticals or biological products.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Biomarkers of Kidney or Renal Transplant Status Materials and Methods

Patients. 45 patients who received a renal transplant in the Nantes University Hospital (Nantes, France) between 1986 and 2007, or who are waiting for one, were retrospectively included in the present study and divided into four groups according to the outcome of their kidney graft. The first group, Transplant Glomerulopathy (TG), comprised 19 patients among whom 6 were in late stage TG and 13 were in early stage TG. The diagnostic of TG is based on the histology of the graft biopsy such as C4d deposits and glomerular basement membrane duplication. The IFTA group was composed of 10 patients who showed evidence of Interstitial Fibrosis and Tubular Atrophy, but no presence of antibody or C4d deposits, and this was also diagnosed histologically after the biopsy. 10 patients who were undergoing dialysis (DIA) while waiting for renal transplantation were included in the third group. The last group was made of renal transplant patients considered as stable (STA), i.e., patients under immunosuppressive treatment showing a stable renal function.

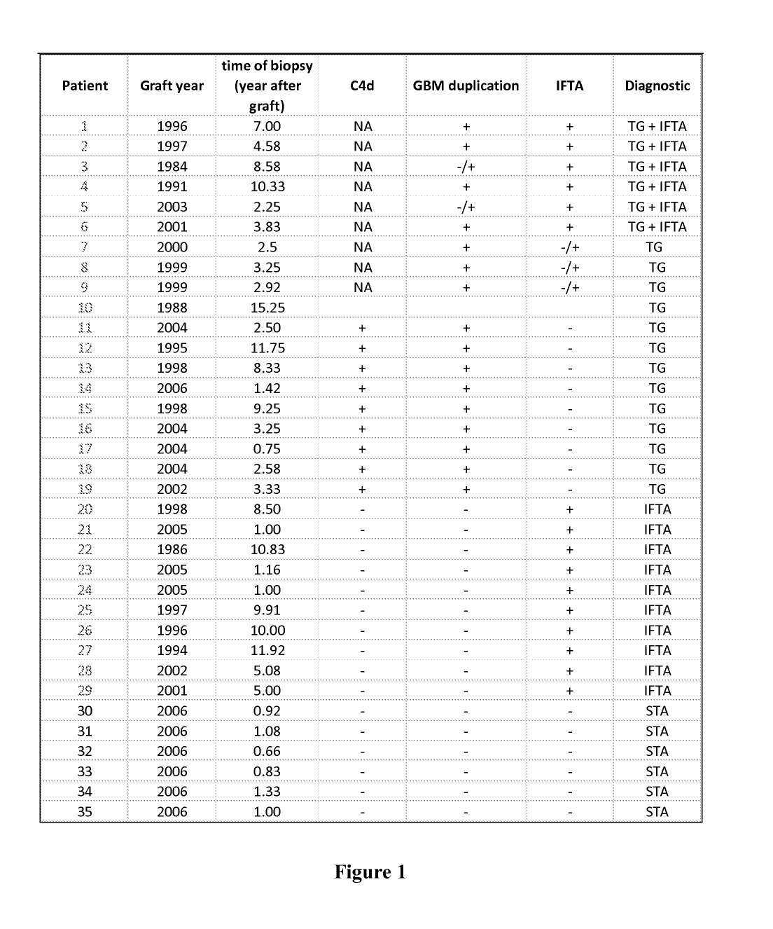

Sera were available from all these patients at the Etablissement Francais du Sang (Nantes, France). Patients' characteristics are summarized in the table presented on FIG. 1. Sera were collected at the time of biopsy from patients of the TG, STA and IFTA groups, and a few days before transplantation from DIA patients. Sera were stored at −80° C. prior to being tested.

Sera Screening on Protein Microarrays (ProtoArrays® Invitrogen). Sera were sent to Invitrogen Corporation (Brandford, Conn., US) for analysis.

Human Protein Collection and Protein Microarray Manufacture. Over 8000 human clones obtained from Invitrogen's Ultimate ORF collection and Gateway collection of kinase clones were transferred into insect cells via baculovirus infection to produce recombinant proteins tagged with Glutathione-S-Transferase (GST). Recombinant proteins were then purified using GST affinity, under native conditions. Because of their production in insect cells and of the particular purification conditions used, the proteins thus obtained are expected to contain appropriate post-translational modifications and to maintain their native conformations. A contact-type printer equipped with 48 matched quill-type pins printed the recombinant proteins in duplicates on a glass slide coated with a thin layer of nitrocellulose.

Protein Microarray Probing. Once printed with duplicates of recombinant proteins and appropriate negative and positive controls, microarray slides were blocked using a blocking buffer containing 50 mM HEPES, 200 mM NaCl, 0.08% Triton X-100, 25% glycerol, 20 mM reduced glutathione, 1.0 mM DTT and 1% BSA, at 4° C. for 1 hour. Microarrays were then probed with a 1:500 dilution of serum sample (one microarray per serum) diluted in 5 mL of freshly prepared PBST buffer and incubated for 90 minutes at 4° C. with gentle agitation. Microarrays were washed five times in 5 mL PBST buffer. Slides were incubated with an Alexa Fluor® 647-conjugated goat anti-human IgG antibody diluted in 5 mL probe buffer to a 1 mg/mL final concentration at 4° C. for 90 minutes. After incubation, slides were washed as described above and dried by spinning in a table top centrifuge equipped with a plate rotor at 1000 rpm for 2 minutes. Arrays were then scanned using an Axon GenePix-4000B fluorescent microarray scanner

Data Acquisition. The GenePix 6.0 software was used for data acquisition. This software overlays the mapping of human proteins in the array list file to each array image with a fixed feature size of 130 μm (diameter). AlexaFluor® 647-conjugated goat anti-human IgG antibody printed on each subarray helps aligning the spots. Other proteins are systematically used as control spots, e.g., anti-biotin antibodies which are recognized by the detection IgG antibody and the Influenza A antigen, which is recognized by approximately 95% of the total population. Scanner settings were selected such that maximal signals on the array were sub-saturated thus ensuring that the full dynamic range of the scanner was utilized.

Data Analysis. Data analysis was performed using Invitrogen's ProtoArray Prospector software. This software follows a 3-step process: (1) Single array analysis: for each protein on each array, a series of values are calculated including background subtracted signals, CI-P value, and replicate spot coefficient of variation; (2) Group characterization: signals for each individual protein across all samples from a given population are aligned for downstream analysis; and (3) Identification of the differences between treated and untreated sample populations. Utilizing M-statistics, proteins are identified for which the differential signals between two populations result in a significant p-value. The output from the comparative analysis includes information on the number of patients in each population that exhibited an immune response against each ProtoArray® protein that was above the M-statistic threshold established for that protein.

Identification of Candidate Biomarkers. All the possible two group-comparisons were performed, which led to lists of total biomarkers for each pathology, at a p-value threshold of 0.05. These lists were compared and biomarkers that were found for more than one pathology were removed from the lists in order to obtain final lists containing only specific biomarkers, i.e., biomarkers unique to one of the 4 pathologies.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1-14. (canceled)

15. An in vitro method for diagnosing or determining kidney status or renal transplant status in a subject, said method comprising steps of:
contacting a biological sample obtained from the subject with at least one biomarker for a time and under conditions allowing a biomarker-antibody complex to form between the at least one biomarker and an antibody present in the biological sample; and
detecting any biomarker-antibody complex formed,
wherein the at least one biomarker is selected from the group consisting of:
a set of biomarkers indicative of kidney status consisting of RAB20, OAS2, IL1RL1(3), GLRA2, SMAD3, ING4(2), CCL19, CENTD2, ECE1, LGALS3, analogs thereof and antibody-binding fragments thereof,
a set of biomarkers indicative of stable renal transplant consisting of ARPC1B, CYP2E1, CDC20, RIT1, PHF21B, RBM34, MS4A6E, C20orf132, LRRFIP1, CKLF(1), analogs thereof and antibody-binding fragments thereof,
a set of biomarkers indicative of renal transplant glomerulopathy consisting of PODN, HM13(4), TSC22D3(2), EPN1, COL23A1, GIMAP6, ZNF174, MAP3K11, CRB3(2), PRKCDBP, DCLK1, FEN1, TANK(2), GLRX5, RAB22A, MAEL, FAM71D, TTC35, analogs thereof, and antibody-binding fragments thereof, and
a set of biomarkers indicative of interstitial fibrosis and tubular atrophy consisting of IL16, MLF1, IFT81(2), DYRK2, NDFIP2, CCNE2, SNX21, TUBB2C, MUC20, ATP1A3, F11R(1), analogs thereof and antibody-binding fragments thereof.

16. The method according to claim 15, wherein the biological sample is contacted with a plurality of biomarkers for a time and under conditions allowing biomarker-antibody complexes to form between the biomarkers and antibodies present in the biological sample.

17. The method according to claim 15, wherein the biological sample is a blood sample.

18. The method according to claim 17, wherein the blood sample is selected from the group consisting of whole blood, serum and plasma.

19. The method according to claim 15, wherein the at least one biomarker is immobilized on a solid carrier or support.

20. The method according to claim 19, wherein the solid carrier or support is an array.

21. The method according to claim 15, wherein detecting any biomarker-antibody complex formed comprises contacting the biological sample with a least one reagent for detecting the biomarker-antibody complex formed between the at least one biomarker and an antibody present in the biological sample.

22. The method according to claim 21, wherein the at least one reagent is a labelled anti-human antibody.

23. The method according to claim 21, wherein detecting the biomarker-antibody complex is performed by immunoassay.

24. A kit for the in vitro diagnostic or determination of kidney status or renal transplant status in a subject, the kit comprising at least one biomarker selected from the group consisting of:
a set of biomarkers indicative of kidney status consisting of RAB20, OAS2, IL1RL1(3), GLRA2, SMAD3, ING4(2), CCL19, CENTD2, ECE1, LGALS3, analogs thereof and antibody-binding fragments thereof,
a set of biomarkers indicative of stable renal transplant consisting of ARPC1B, CYP2E1, CDC20, RIT1, PHF21B, RBM34, MS4A6E, C20orf132, LRRFIP1, CKLF(1), analogs thereof and antibody-binding fragments thereof,
a set of biomarkers indicative of renal transplant glomerulopathy consisting of PODN, HM13(4), TSC22D3(2), EPN1, COL23A1, GIMAP6, ZNF174, MAP3K11, CRB3(2), PRKCDBP, DCLK1, FEN1, TANK(2), GLRX5, RAB22A, MAEL, FAM71D, TTC35, analogs thereof, and antibody-binding fragments thereof, and
a set of biomarkers indicative of interstitial fibrosis and tubular atrophy consisting of IL16, MLF1, IFT81(2), DYRK2, NDFIP2, CCNE2, SNX21, TUBB2C, MUC20, ATP1A3, F11R(1), analogs thereof and antibody-binding fragments thereof.

25. The kit according to claim 24, wherein the kit comprises a plurality of biomarkers.

26. The kit according to claim 24, wherein the at least one biomarker is immobilized on a solid carrier or support.

27. The kit according to claim 26, wherein the solid carrier or support is an array.

28. The kit according to claim 24, further comprising at least one reagent for detecting a biomarker-antibody complex formed between the at least one biomarker included in the kit and an antibody present in a biological sample obtained from a subject.

29. The kit according to claim 28, wherein the at least one reagent is a labeled anti-human antibody.

30. An array for the in vitro diagnostic or determination of kidney status or renal transplant status in a subject, the array consisting essentially of a plurality of biomarkers, attached to its surface, wherein the plurality of biomarkers are selected from the group consisting of:
a set of biomarkers indicative of kidney status consisting of RAB20, OAS2, IL1RL1(3), GLRA2, SMAD3, ING4(2), CCL19, CENTD2, ECE1, LGALS3, analogs thereof and antibody-binding fragments thereof,
a set of biomarkers indicative of stable renal transplant consisting of ARPC1B, CYP2E1, CDC20, RIT1, PHF21B, RBM34, MS4A6E, C20orf132, LRRFIP1, CKLF(1), analogs thereof and antibody-binding fragments thereof,
a set of biomarkers indicative of renal transplant glomerulopathy consisting of PODN, HM13(4), TSC22D3(2), EPN1, COL23A1, GIMAP6, ZNF174, MAP3K11, CRB3(2), PRKCDBP, DCLK1, FEN1, TANK(2), GLRX5, RAB22A, MAEL, FAM71D, TTC35, analogs thereof, and antibody-binding fragments thereof, and
a set of biomarkers indicative of interstitial fibrosis and tubular atrophy consisting of IL16, MLF1, IFT81(2), DYRK2, NDFIP2, CCNE2, SNX21, TUBB2C, MUC20, ATP1A3, F11R(1), analogs thereof and antibody-binding fragments thereof.
